(19) United States
(12) Patent Application Publication
(10) Pub. No.: US 2021/0290509 A1
(43) Pub. Date: Sep. 23, 2021

(54) IMPROVED CONDITIONING HAIR TREATMENT PRODUCT WITH WASHOUT PROTECTION

(71) Applicant: Henkel AG & Co. KGaA, Duesseldorf (DE)

(72) Inventors: VOLKER SCHEUNEMANN, Lueneburg (DE); ERIK SCHULZE ZUR WIESCHE, Hamburg (DE); RENE KROHN, Norderstedt (DE)

(73) Assignee: Henkel AG & Co. KGaA, Duesseldorf (DE)

(21) Appl. No.: 16/336,845

(22) PCT Filed: Jul. 19, 2017
(86) PCT No.: PCT/EP2017/068237

§ 371 Date: Mar. 26, 2019

(30) Foreign Application Priority Data
Sep. 30, 2016 (DE) 10 2016 218 998.0

(51) Int. Cl. A61K 8/44; A61K 8/365; A61Q 5/00

(52) U.S. Cl. CPC A61K 8/44; A61Q 17/005; A61Q 5/004; A61K 8/365

(57) ABSTRACT

Hair treatment products contain 0.001 to 20% by weight of a complexing agent selected from tetrasodium-N,N-bis(carboxylatomethyl)-L-glutamate, pentasodium diethylenetriamine pentaacetic acid (DTPA), tetrasodium disuccinate (IDS) and from about 0.05 to about 0.5 mol/kg of either a bivalent salt of a monovalent organic acid or 0.01 to 0.5 mol/kg of at least one monovalent organic acid (c1) and 0.01 to 0.1 mol/kg at least one bivalent inorganic salt (c2). Use improves structural reinforcement and reduces or prevents color washout in dyed hair.

### CROSS-REFERENCE TO RELATED APPLICATION

This application is a U.S. National-Stage entry under 35 U.S.C. § 371 based on International Application No. PCT/EP2017/068237, filed Jul. 19, 2017, which was published under PCT Article 21(2) and which claims priority to German Application No. 102016218 998.0, filed Sep. 30, 2016, which are all hereby incorporated in their entirety by reference.

### TECHNICAL FIELD

The present disclosure relates to hair treatment agents, in particular shampoos and what are referred to as conditioners, having an active ingredient combination for the gentle and effective care of hair.

### BACKGROUND

The importance of care products with the longest-lasting possible effect is increasing not least due to the high amount of strain on hair caused, for example, by coloring or permanents, by cleaning hair with shampoos, and by environmental pressures. Care products of this type influence the natural structure and properties of hair. For example, the wet and dry combability, the hold and the volume of hair can be optimized following care treatments of this kind, or hair can be protected from increased splitting.

It has long been customary, therefore, to subject hair to a special aftertreatment. In this process, the hair is treated with special active ingredients, for example quaternary ammonium salts or special polymers, usually in the form of a rinse. As a result of this treatment, depending on the formulation, the combability, the hold, and the volume of the hair are improved, and the amount of splitting is reduced.

Multi-functional cosmetic products are also known in the prior art. These include in particular what are referred to as “2 in 1” shampoos, which not only clean hair but also condition it. Products of this type are held in high esteem by consumers because, due to their product performance, they eradicate the need for at least one process step, for example conditioning with a conventional hair conditioner.

Similarly, products for modifying natural hair color play a prominent role in hair cosmetics. A distinction is made between permanent, semi-permanent, or temporary coloring systems based on chemical and/or natural dyes. Hair colors produced artificially by permanent, semi-permanent or temporary coloring systems are however disadvantageous in that they can change undesirably, for example during or after hair cleaning.

An “undesirable change” is understood to mean fading or bleeding and loss of the color brilliance of the hair shade achieved by the particular coloring. Environmental impacts and/or the effects of sunlight can further intensify these changes.

There continues to be a need to provide active ingredients or active ingredient combinations for hair treatment agents which have good nourishing properties and also strengthen the adhesion of dyes to hair fibers and thus maintain the fastness of the artificially produced hair color, and in this respect to develop hair treatment agents.

It has been observed, however, that water hardness can sometimes negatively influence the nourishing properties of hair treatment agents, and therefore the nourishing performance of the same product may be either too low or too high depending on water quality.

Nourishing performance that is too high or “over-nourishment” (occurrence of what is known as a build-up effect) of the hair is understood to mean in particular a greasy hair feel, lack of hair volume, and/or a lank, unkempt appearance of the hair, whereas damaged hair (broken and dull hair that is prone to splitting) is an indicator of too little nourishing performance.

### BRIEF SUMMARY

A hair treatment agent contains, based on its weight,

a) from about 0.001 to about 20 wt. % of complexing agents selected from the group consisting of
a. tetrasodium-N,N-bis(carboxylatomethyl)-L-glutamate (tetrasodium glutamate diacetate, GLDA)

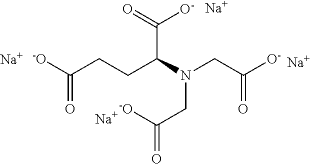

b. pentasodium diethylenetriaminepentaacetate (DTPA)

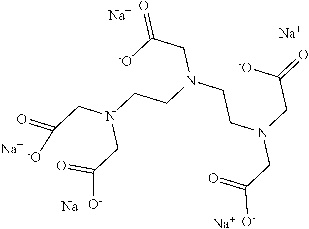

c. tetrasodium iminodisuccinate (IDS)

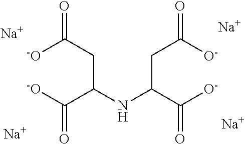

and either

b) from about 0.05 to about 0.5 mol/kg of at least one divalent salt of a monovalent organic acid or

c) from about 0.01 to about 0.5 mol/kg of at least one monovalent organic acid (c1), and from about 0.01 to about 0.1 mol/kg of at least one divalent inorganic salt (c2).

A method for treating hair comprises applying such an agent to dry or wet hair, leaving the applied agent on the hair for a period of ten seconds or longer, and rinsing the agent out thereafter. In various embodiments, the agent is left on the hair for a period up to about 300 seconds, or is left on the hair until the next ashing.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object of the present application was therefore to provide skin-compatible, nourishing hair treatment agents which have excellent foaming properties, can be rinsed out well with water and additionally reduce or prevent washout of color from colored hair.

The nourishing hair treatment agents should have a uniform conditioning performance irrespective of water quality and in particular should not weigh down fine hair and/or damaged hair, but should strengthen the structure of the hair and thus protect it from splitting and breaking and should improve combability and the feel of the hair. In addition, said agents should have a germicidal effect.

It has now been found that a combination of certain ingredients has a particularly positive effect on colored hair and the hair follicles treated with said ingredients.

A first subject of the present disclosure is hair treatment agents containing, based on their weight,
a) from about 0.001 to about 20 wt. % of complexing agents selected from tetras odium-N,N-bis (carboxylatomethyl)-L-glutamate (tetrasodium glutamate diacetate, GLDA)

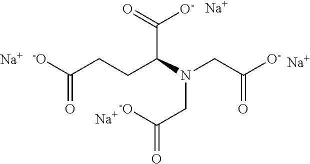

pentasodium diethylenetriaminepentaacetate (DTPA)

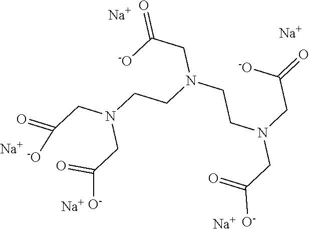

tetrasodium iminodisuccinate (IDS)

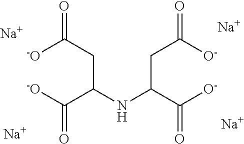

and either
b) from about 0.05 to about 0.5 mol/kg of at least one divalent salt of a monovalent organic acid or
c) from about 0.01 to about 0.5 mol/kg of at least one monovalent organic acid (c1), and from about 0.01 to about 0.1 mol/kg of at least one divalent inorganic salt (c2).

Hair treatment agents within the meaning of the present disclosure are, for example, hair shampoos, hair conditioners, conditioning shampoos, hair sprays, hair rinses, hair masques, hair packs, hair tonics, permanent wave fixing solutions, hair coloring shampoos, hair dyes, hair setting lotions, hair setting products, hair styling preparations, blow-dry wave lotions, styling mousses, hair gels, hair waxes, or combinations thereof. In view of the fact that men in particular are often reluctant to use multiple different agents and/or multiple application steps, agents that men use already are preferred. Preferred agents are therefore shampoos, conditioning agents, or hair tonics.

The hair treatment agents contain, as a first essential ingredient, from about 0.001 to about 20 wt. % of complexing agents selected from the above-mentioned group. The agents may contain only one of the three complexing agents mentioned. However, it is also possible for the agents to contain two or all three of the above-mentioned complexing agents, the amount of all the complexing agents from the above-mentioned group contained in the agents being within an amount range of from about 0.01 to about 20 wt. %.

Hair treatment agents which are preferred as contemplated herein contain, based on their weight, from about 0.005 to about 15 wt. %, preferably from about 0.01 to about 10 wt. %, more preferably from about 0.05 to about 7.5 wt. %, and in particular from about 0.1 to about 5 wt. %, of tetrasodium-N,N-bis(carboxylatomethyl)-L-glutamate (tetrasodium glutamate diacetate, GLDA).

Hair treatment agents which are also preferred contain, based on their weight, from about 0.005 to about 15 wt. %, preferably from about 0.01 to about 10 wt. %, more preferably from about 0.05 to about 7.5 wt. %, and in particular from about 0.1 to about 5 wt. %, of pentasodium diethylenetriaminepentaacetate (DTPA).

Hair treatment agents which are also preferred as contemplated herein contain, based on their weight, from about 0.005 to about 15 wt. %, preferably from about 0.01 to about 10 wt. %, more preferably from about 0.05 to about 7.5 wt. %, and in particular from about 0.1 to about 5 wt. %, of tetrasodium iminodisuccinate (IDS).

Hair treatment agents which are also preferred as contemplated herein contain, based on their weight, from about 0.01 to about 10 wt. %, more preferably from about 0.05 to about 7.5 wt. %, and in particular from about 0.1 to about 5 wt. %, of tetrasodium-N,N-bis(carboxylatomethyl)-L-glutamate (tetrasodium glutamate diacetate, GLDA), and from about 0.01 to about 10 wt. %, more preferably from about 0.05 to about 7.5 wt. %, and in particular from about 0.1 to about 5 wt. %, of pentasodium diethylenetriaminepentaacetate (DTPA).

Hair treatment agents which are also preferred as contemplated herein contain, based on their weight, from about 0.01 to about 10 wt. %, more preferably from about 0.05 to about 7.5 wt. %, and in particular from about 0.1 to about 5 wt. %, of tetrasodium-N,N-bis(carboxylatomethyl)-L-glutamate (tetrasodium glutamate diacetate, GLDA), and from about 0.01 to about 10 wt. %, more preferably from about 0.05 to about 7.5 wt. %, and in particular from about 0.1 to about 5 wt. %, of tetrasodium iminodisuccinate (IDS).

Hair treatment agents which are also preferred as contemplated herein contain, based on their weight, from about 0.01 to about 10 wt. %, more preferably from about 0.05 to about 7.5 wt. %, and in particular from about 0.1 to about 5 wt. %, of pentasodium diethylenetriaminepentaacetate (DTPA), and from about 0.01 to about 10 wt. %, more preferably from about 0.05 to about 7.5 wt. %, and in particular from about 0.1 to about 5 wt. %, of tetrasodium iminodisuccinate (IDS).

Hair treatment agents which are also preferred as contemplated herein contain, based on their weight, from about 0.1 to about 5 wt. % of tetrasodium-N,N-bis(carboxylatomethyl)-L-glutamate (tetrasodium glutamate diacetate, GLDA), from about 0.1 to about 5 wt. % of pentasodium diethylenetriaminepentaacetate (DTPA), and from about 0.1 to about 5 wt. % of tetrasodium iminodisuccinate (IDS).

Suitable bivalent salts (component b)), which can be used in the hair treatment agents, are preferably selected from alkaline-earth metal and/or zinc salts of the monovalent organic acids from the group formed of formic acid, acetic acid, glycolic acid, gluconic acid, lactic acid, mandelic acid, and/or glyoxylic acid.

Particularly suitable salts b) are calcium, magnesium and/or zinc salts of the above-mentioned acids. Calcium salts, such as calcium gluconate and calcium lactate, are very particularly preferred. Calcium lactate is particularly preferred.

Hair treatment agents which are also preferred contain, as component b), salts from the group of alkaline-earth metal and/or zinc salts of formic acid, acetic acid, glycolic acid, gluconic acid, lactic acid, mandelic acid and/or glyoxylic acid, or, as component (c1), formic acid, acetic acid, glycolic acid, gluconic acid, lactic acid, mandelic acid and/or glyoxylic acid, and, as component (c2), an alkaline-earth metal halide, alkaline-earth metal hydroxide, alkaline-earth metal sulfate, alkaline-earth metal oxide, zinc halide, zinc hydroxide, zinc sulfate, and/or zinc oxide.

Suitable components c), which can be used in the cosmetic compositions of the method as contemplated herein, are preferably selected from monovalent organic acids from the group formed of formic acid, acetic acid, glycolic acid, gluconic acid, lactic acid, mandelic acid, and/or glyoxylic acid (c1), and from bivalent inorganic salts selected from the group formed of alkaline-earth metal halides, alkaline-earth metal hydroxides, alkaline-earth metal sulfates, alkaline-earth metal oxides, zinc halides, zinc hydroxides, zinc sulfates, and/or zinc oxides (c2).

Particularly suitable acids (c1) are lactic acid and gluconic acid. Lactic acid is particularly suitable.

Particularly suitable salts (c2) are calcium, magnesium and/or zinc halides, and calcium, magnesium and/or zinc hydroxides. Calcium, magnesium and/or zinc chloride and calcium, magnesium and/or zinc hydroxide are very particularly preferred. Calcium chloride and calcium hydroxide are particularly preferred.

Hair treatment agents which are preferred as contemplated herein contain,
as component b), calcium lactate or,
as component (c1), lactic acid and, as component (c2), calcium chloride and/or calcium hydroxide.

Hair treatment agents which are particularly preferred as contemplated herein contain
from about 0.04 to about 0.40 mol/kg, preferably from about 0.06 to about 0.30 mol/kg, and in particular from about 0.07 to about 0.20 mol/kg, of salt b) or
from about 0.02 to about 0.40 mol/kg, preferably from about 0.03 to about 0.30 mol/kg, and in particular from about 0.05 to about 0.20 mol/kg, of the monovalent organic acid (c1), and from about 0.04 to about 0.10 mol/kg, preferably from about 0.05 to about 0.09 mol/kg, and in particular from about 0.06 to about 0.08 mol/kg, of the divalent organic acid (c2).

It has been found that the agents as contemplated herein are particularly effective and the color change of the artificially produced hair color after several cleaning processes is particularly low if the hair treatment agents contain calcium lactate or a mixture of lactic acid and calcium chloride and/or calcium hydroxide and have a pH in the range of from about 3.0 to about 5.0, preferably in the range of from about 4.1 to about 4.75, more preferably from about 4.2 to about 4.7, and in particular from about 4.3 to about 4.6. Hair treatment agents which are particularly preferred as contemplated herein have a pH in the range of from about 3.0 to about 5.0, preferably in the range of from about 4.1 to about 4.75, more preferably from about 4.2 to about 4.7, and in particular from about 4.3 to about 4.6.

The agents can of course be applied to any hair. They are fundamentally advantageous on any hair that has been colored using permanent, semi-permanent or temporary hair colors. Temporary hair colors are, however, intended to be washed out and/or to fade over time, and therefore agents as contemplated herein are particularly suitable for use on hair that has been colored using permanent or oxidative hair dyes.

The agents may contain amino-functional silicones to further increase the conditioning effects. Preferred agents as contemplated herein contain from about 0.001 to about 50 wt. % of at least one amino-functional silicone.

Particularly preferred agents as contemplated herein contain at least one amino-functional silicone of formula (Si-IIa)

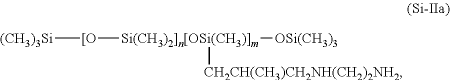

in which m and n are numbers whose sum (m+n) is between about 1 and about 2000, preferably between about 50 and about 150, n preferably assuming values of from 0 to about 1999 and in particular from about 49 to about 149 and m preferably assuming values of from about 1 to about 2000, in particular from about 1 to about 10.

These silicones are designated as trimethylsilylamodimethicones in accordance with the INCI declaration.

Particularly preferred as well are agents as contemplated herein that contain an amino-functional silicone of formula (Si-IIb)

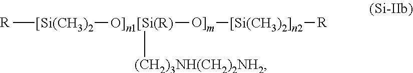

in which R represents —OH, —O—CH₃, or a —CH₃ group and m, n1 and n2 are numbers whose sum (m+n1+n2) is between 1 and about 2000, preferably between about 50 and about 150, the sum (n1+n2) preferably assuming values of from 0 to about 1999 and in particular from about 49 to about 149 and m preferably assuming values of from 1 to about 2000, in particular from 1 to about 10.

These silicones are designated as amodimethicones in accordance with the INCI declaration.

Irrespective of which amino-functional silicones are used, agents as contemplated herein are preferred that contain an amino-functional silicone of which the amine value is above about 0.25 meq/g, preferably above about 0.3 meq/g, and in particular above about 0.4 meq/g. The amine value here represents the milliequivalents of amine per gram of the amino-functional silicone. Said value can be determined by titration and may also be given in the unit mg KOH/g.

Hair treatment agents which are preferred as contemplated herein contain, based on their weight, from about 0.01 to about 20 wt. %, preferably from about 0.05 to about 10 wt. %, more preferably from about 0.1 to about 7.5 wt. %, and in particular from about 0.15 to about 5 wt. %, of amino-functional silicone(s). The agents as contemplated herein particularly preferably contain amino-functional silicone(s) having terminal hydroxy group(s). Some special amino-functional silicone(s) having terminal hydroxy group(s) have been found to be particularly suitable in the agents as contemplated herein. These are described in the following.

Pretreatment agents that contain at least one silicone of formula (Si-V) have been found to be particularly effective in the method as contemplated herein with regard to the desired effects:

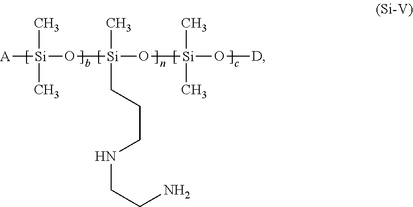

in which

A represents an —OH, —O—Si(CH₃)₃, —O—Si(CH₃)₂OH, —O—Si(CH₃)₂OCH₃ group,

D represents an —H, —Si(CH₃)₃, —Si(CH₃)₂OH, −Si(CH₃)₂OCH₃ group,

b, n and c represent integers between 0 and about 1000, with the proviso that
n>0 and b+c>0
at least one of the conditions A=—OH or D=−H is met.

Hair treatment agents as contemplated herein are therefore preferred that contain, based on their weight, from about 0.01 to about 20 wt. %, preferably from about 0.01 to about 20 wt. %, more preferably from about 0.1 to about 10 wt. %, even more preferably from about 0.5 to about 7.5 wt. %, and in particular from about 0.1 to about 5 wt. %, of at least one silicone of formula (Si-V):

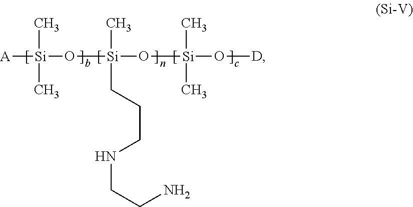

in which
A represents an —OH, —O—Si(CH₃)₃, —O—Si(CH₃)₂OH, or —O—Si(CH₃)₂OCH₃ group,
D represents an —H, —Si(CH₃)₃, —Si(CH₃)₂OH, —Si(CH₃)₂OCH₃ group,
b, n and c represent integers between 0 and 1000, with the proviso that
   n>0 and b+c>0
   at least one of the conditions A=—OH or D=—H is met.

In the above formula (Si-V), the individual siloxane units having the indices b, c and n are randomly distributed, i.e. they are not necessarily block copolymers.

Further particularly suitable silicones are 4-morpholinomethyl-substituted. Hair treatment agents as contemplated herein are particularly preferred that contain, based on their weight, from about 0.001 to about 20 wt. %, preferably from about 0.01 to about 10 wt. %, particularly preferably from about 0.05 to about 7.5 wt. %, and in particular from about 0.5 to about 5 wt. %, of at least one 4-morpholinomethyl-substituted silicone which comprises structural units of formulas (Si-Via), (Si-VIb) and (Si-Vic)

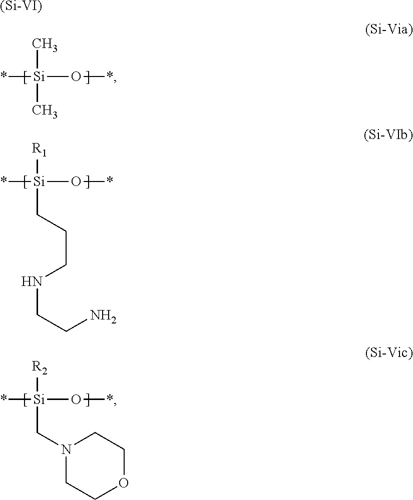

in which

R1 represents —CH₃, —OH, —OCH₃, —O—CH₂CH₃, —O—CH₂CH₂CH₃, or —O—CH(CH₃)₂;

R2 represents —CH₃, —OH, or —OCH₃.

Particularly preferred hair treatment agents as contemplated herein contain, based on their weight, from about 0.001 to about 20 wt. %, preferably from about 0.01 to about 10 wt. %, particularly preferably from about 0.05 to about 7.5 wt. %, and in particular from about 0.5 to about 5 wt. %, of at least one 4-morpholinomethyl-substituted silicone of formula (Si-VI)

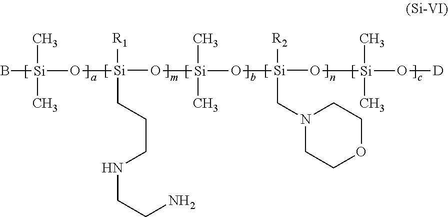

in which
R1 represents —CH₃, —OH, —OCH₃, —O—CH₂CH₃, —O—CH₂CH₂CH₃, or —O—CH(CH₃)₂,
R2 represents —CH₃, —OH, or —OCH₃;
B represents an —OH, —O—Si(CH₃)₃, —O—Si(CH₃)₂OH, or —O—Si(CH₃)₂OCH₃ group;
D represents an —H, —Si(CH₃)₃, —Si(CH₃)₂OH, —Si(CH₃)₂OCH₃ group,
a, b and c represent, independently of one another, integers between 0 and about 1000, with the proviso that a+b+c>0;
m and n represent, independently of one another, integers between about 1 and about 1000, with the proviso that
   at least one of the conditions B=—OH or D=—H is met,
   the units a, b, c, m and n are distributed randomly or in blocks in the molecule.

Structural formula (Si-VI) is intended to indicate that the siloxane groups n and m do not necessarily have to be directly bonded to an end group B or D. Instead, in preferred formulas (Si-VI), a>0 or b>0 and, in particularly preferred formulas (Si-VI), a>0 and c>0; i.e. the terminal group B or D is preferably bonded to a dimethylsiloxy group. In formula (Si-VI) as well, the siloxane units a, b, c, m and n are preferably distributed randomly.

The silicones represented by formula (Si-VI) and used as contemplated herein can be trimethylsilyl-terminated (D or B=—Si(CH₃)₃), but they may also be dimethylsilylhydroxy-terminated at both ends or dimethylsilylhydroxy-terminated at one end and dimethylsilylmethoxy-terminated at the other end. In the context of the present disclosure, silicones used particularly preferably are selected from silicones in which

B = —O—Si(CH₃)₂OH and D = —Si(CH₃)₃
B = —O—Si(CH₃)₂OH and D = —Si(CH₃)₂OH
B = —O—Si(CH₃)₂OH and D = —Si(CH₃)₂OCH₃
B = —O—Si(CH₃)₃ and D = —Si(CH₃)₂OH
B = —O—Si(CH₃)₂OCH₃ and D = —Si(CH₃)₂OH.

These silicones lead to enormous improvements in the hair properties of hair treated with the agents as contemplated herein, and to greatly improved protection during oxidative treatment.

Irrespective of the type of amino-functional silicone(s) having terminal hydroxy group(s) used, the agents as contemplated herein contain the silicone(s) preferably in the form of an emulsion, particularly preferably in the form of a microemulsion.

It has been found that the effect of the silicones used in the agents as contemplated herein can be increased further still if certain nonionic components are also used in the agents. In addition, these nonionic components have positive effects on the storage stability of the agents. Nonionic components that are particularly suitable here are ethoxylates of decanol, undecanol, dodecanol, tridecanol, etc. Ethoxylated tridecanols which are particularly preferably incorporated in the agents as contemplated herein have been found to be particularly suitable. Agents which are particularly preferred as contemplated herein contain, based on their weight, from about 0.00001 to about 5 wt. %, preferably from about 0.0001 to about 3.5 wt. %, particularly preferably from about 0.001 to about 2 wt. %, more preferably from about 0.01 to about 1 wt. %, and in particular from about 0.1 to about 0.5 wt. %, of branched ethoxylated tridecanol (INCI name: trideceth-5) or α-iso-tridecyl-w-hydroxy polyglycol ether (INCI name: trideceth-10) or mixtures thereof.

In addition to or instead of the amino-functional silicone(s), the agents as contemplated herein may contain further silicone(s) which is/are not amino-functional. Preferred agents as contemplated herein contain at least one silicone selected from:
(i) polyalkylsiloxanes, polyarylsiloxanes, polyalkylarylsiloxanes, which are volatile or non-volatile, straight-chain, branched or cyclic, crosslinked or not crosslinked;
(ii) polysiloxanes which contain one or more organofunctional groups in their general structure, said groups being selected from:
   a) (per)fluorinated groups;
   b) thiol groups;
   c) carboxylate groups;
   d) hydroxylated groups;
   e) alkoxylated groups;
   f) acyloxyalkyl groups;
   g) amphoteric groups;
   h) bisulfite groups;
   i) hydroxyacylamino groups;
   j) carboxyl groups;
   k) sulfonic acid groups; and
   l) sulfate or thiosulfate groups;
(iii) linear polysiloxane (A)-polyoxyalkylene (B) block copolymers of type (A-B)ₙ, where n>3;
(iv) grafted silicone polymers which have a silicone-free organic backbone and which of an organic main chain, which is formed from organic monomers that do not contain silicone and onto which at least one polysiloxane macromer has been grafted in the chain and also optionally at at least one chain end;
(v) grafted silicone polymers which have a polysiloxane backbone, onto which silicone-free organic monomers have been grafted which comprise a polysiloxane main chain onto which at least one organic macromer that does not contain silicone has been grafted in the chain and also optionally at at least one of the ends thereof;
   or mixtures thereof.

Hair treatment agents which are preferred as contemplated herein contain, based on their weight, from about 0.01 to about 20 wt. %, preferably from about 0.1 to about 10 wt. %, more preferably from about 0.5 to about 7.5 wt. %, and in particular from about 1 to about 5 wt. %, of non-amino-functional silicone(s). Preferred silicones are described in the following.

Particularly preferred agents as contemplated herein contain at least one silicone of formula Si-I

(CH₃)₃Si—[O—Si(CH₃)₂]ₓ—O—Si(CH₃)₃ (Si-I),

in which x represents a number from 0 to about 100, preferably from 0 to about 50, more preferably from 0 to about 20, and in particular from 0 to about 10.

These silicones are referred to in accordance with INCI nomenclature as DIMETHICONES. The following compounds are preferably used within the scope of the present disclosure as a silicone of formula Si-I:

(CH₃)₃Si—O—Si(CH₃)₃ (CH₃)₃Si—O—(CH₃)₂Si—O—Si(CH₃)₃ (CH₃)₃Si—[O—(CH₃)₂Si]²—O—Si(CH₃)₃ (CH₃)₃Si—[O—(CH₃)₂Si]₃—O—Si(CH₃)₃(CH₃)₃Si—[O—(CH₃)₂Si]₄—O—Si(CH₃)₃ (CH₃)₃Si—[O—(CH₃)₂Si]₅—O—Si(CH₃)₃(CH₃)₃Si—[O—(CH₃)₂Si]₆—O—Si(CH₃)₃ (CH₃)₃Si—[O—(CH₃)₂Si]₇—O—Si(CH₃)₃(CH₃)₃Si—[O—(CH₃)₂Si]₈—O—Si(CH₃)₃ (CH₃)₃Si—[O—(CH₃)₂Si]₉—O—Si(CH₃)₃(CH₃)₃Si—[O—(CH₃)₂Si]₁₀—O—Si(CH₃)₃ (CH₃)₃Si—[O—(CH₃)₂Si]₁₁—O—Si(CH₃)₃(CH₃)₃Si—[O—(CH₃)₂Si]₁₂—O—Si(CH₃)₃ (CH₃)₃Si—[O—(CH₃)₂Si]₁₃—O—Si(CH₃)₃(CH₃)₃Si—[O—(CH₃)₂Si]₁₄—O—Si(CH₃)₃ (CH₃)₃Si—[O—(CH₃)₂Si]₁₅—O—Si(CH₃)₃(CH₃)₃Si—[O—(CH₃)₂Si]¹⁶—O—Si(CH₃)₃ (CH₃)₃Si—[O—(CH₃)₂Si]₁₇—O—Si(CH₃)₃(CH₃)₃Si—[O—(CH₃)₂Si]₁₈—O—Si(CH₃)₃ (CH₃)₃Si—[O—(CH₃)₂Si]₁₉—O—Si(CH₃)₃(CH₃)₃Si—[O—(CH₃)₂Si]₂₀—O—Si(CH₃)₃ (CH₃)₃Si—O—Si(CH₃)₃, (CH₃)₃Si—O—(CH₃)₂Si—O—Si(CH₃)₃ and/or (CH₃)₃Si—[O—(CH₃)₂Si]₂—O—Si(CH₃)₃ being particularly preferred.

Of course, mixtures of the above-mentioned silicones can also be contained in the agents as contemplated herein. Preferred silicones that can be used as contemplated herein have viscosities at about 20° C. of from about 0.2 to about 2 mm²s⁻¹, silicones having viscosities of from about 0.5 to about 1 mm²s⁻¹ being particularly preferred. The hair treatment agents as contemplated herein may contain surfactant(s). In cleaning compositions (shampoos), in particular anionic surfactants have been found to be suitable and, in conditioning compositions, cationic surfactants are ingredients that are often used; owing to their advantageous properties, amphoteric surfactants are particularly preferably used in both shampoos and conditioners.

The hair treatment agents as contemplated herein may contain at least one anionic surfactant.

Suitable anionic surfactants and emulsifiers for the compositions as contemplated herein include all anionic surface-active substances suitable for use on the human body. These are exemplified by a water-solubilizing, anionic group such as a carboxylate, sulfate, sulfonate or phosphate group and a lipophilic alkyl group having approximately 8 to 30 C atoms. In addition, glycol or polyglycol ether groups, ester, ether and amide groups, and hydroxyl groups may be contained in the molecule.

Preferred anionic surfactants and emulsifiers are acyl glutamates, acyl isethionates, acyl sarcosinates and acyl taurates, each having a linear or branched acyl functional group having from about 6 to about 22 carbon atoms and 0, 1, 2 or 3 double bonds, which in particularly preferred embodiments is selected from an octanoyl, decanoyl, lauroyl, myristoyl, palmitoyl and stearoyl functional group, esters of tartaric acid, citric acid or succinic acid, or the salts of said acids with alkylated glucose, in particular the products having the INCI name disodium coco-glucoside citrate, sodium coco-glucoside tartrate and disodium coco-glucoside sulfosuccinate, alkyl polyglycol ether sulfates and ether carboxylic acids having from about 8 to about 18 C atoms in the alkyl group and up to about 12 ethoxy groups in the molecule, sulfosuccinic acid monoalkyl and dialkyl esters having from about 8 to about 18 C atoms in the alkyl group and sulfosuccinic acid monoalkyl polyoxyethyl esters having from about 8 to about 18 C atoms in the alkyl group and from about 1 to about 6 ethoxy groups.

Further preferred anionic surfactants are alkyl sulfates, alkyl polyglycol ether sulfates and ether carboxylic acid salts having from about 10 to about 18 C atoms in the alkyl group and up to about 12 glycol ether groups in the molecule, and sulfosuccinic acid mono and dialkyl esters having from about 8 to about 18 C atoms in the alkyl group, and sulfosuccinic acid monoalkyl polyoxyethyl esters having from about 8 to about 18 C atoms in the alkyl group and from about 1 to about 6 oxyethyl groups.

Particularly preferred anionic surfactants are the alkali or ammonium salts of lauryl ether sulfate having a degree of ethoxylation of from about 2 to about 4 EO.

Preferred hair treatment agents contain, based on their weight, from about 0.5 to about 20 wt. %, preferably from about 0.75 to about 15 wt. %, more preferably from about 1 to about 12 wt. %, and in particular from about 2 to about 10 wt. %, of anionic surfactant(s).

Particularly preferred hair treatment agents as contemplated herein contain, based on their weight, from about 0.5 to about 20 wt. %, preferably from about 0.75 to about 15 wt. %, more preferably from about 1 to about 12 wt. %, and in particular from about 2 to about 10 wt. %, of alkyl (ether) sulfates of general formula R—(OCH₂—CH₂)ₙ—OSO₃X, in which R is a straight-chain or branched, saturated or unsaturated alkyl group having from about 8 to about 24 C atoms, n is the number 0 or from about 1 to about 12, and X is an alkali, alkaline-earth, ammonium or alkanolamine ion.

The hair treatment agents as contemplated herein may contain at least one amphoteric surfactant and/or at least one nonionic surfactant.

Particularly preferred hair treatment agents as contemplated herein contain, based on their weight, from about 0.3 to about 20 wt. %, preferably from about 0.5 to about 8 wt. %, more preferably from about 0.75 to about 6 wt. %, and in particular from about 1 to about 5 wt. %, of amphoteric surfactant(s). Surfactants that have both a negatively-charged functional group and a positively-charged functional group are referred to as amphoteric surfactants or zwitterionic surfactants.

Particularly suitable zwitterionic surfactants are what are referred to as betaines, such as N-alkyl-N,N-dimethylammonium glycinates, for example cocoalkyl dimethylammonium glycinate, N-acylaminopropyl-N,N-dimethylammonium glycinates, for example cocoacylaminopropyl dimethylammonium glycinate, 2-alkyl-3-carboxymethyl-3-hydroxyethyl imidazolines each having from about 8 to about 18 C atoms in the alkyl or acyl group, and cocoacylaminoethyl hydroxyethyl carboxymethyl glycinate. A preferred zwitterionic surfactant is the fatty acid amide derivative known by the INCI name cocamidopropyl betaine.

Further examples of suitable ampholytic surfactants are N-alkylglycines, N-alkylaminopropionic acids, N-alkylaminobutyric acids, N-alkyliminodipropionic acids, N-hydroxyethyl-N-alkyl amidopropyl glycines, N-alkyl taurines, N-alkyl sarcosines, 2-alkylaminopropionic acids, and alkylaminoacetic acids having in each case approximately 8 to 24 C atoms in the alkyl group. Particularly preferred ampholytic surfactants are N-cocoalkylaminopropionate, cocoacylaminoethyl-aminopropionate, and C₁₂-C₁₈ acylsarcosine.

Particularly preferred hair treatment agents contain, as amphoteric surfactants, betaines of formula (Bet-I)

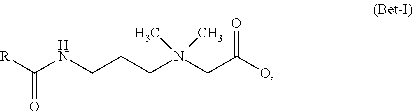

in which R represents a straight-chain or branched, saturated or mono or polyunsaturated alkyl or alkenyl functional group having from about 8 to about 24 carbon atoms.

In accordance with the INCI nomenclature, these surfactants are referred to as amidopropyl betaines, the representatives derived from coconut fatty acids being preferred and referred to as cocamidopropyl betaines. It is particularly preferable as contemplated herein to use surfactants of formula (Bet-I) that are a mixture of the following representatives:

H₃C—(CH₂)₇—C(O)—NH—(CH₂)₃N⁺(CH₃)₂CH₂COO⁻
H₃C—(CH₂)₉—C(O)—NH—(CH₂)₃N⁺(CH₃)₂CH₂COO⁻
H₃C—(CH₂)₁₁—C(O)—NH—(CH₂)₃N⁺(CH₃)₂CH₂COO⁻
H₃C—(CH₂)₁₃—C(O)—NH—(CH₂)₃N⁺(CH₃)₂CH₂COO⁻
H₃C—(CH₂)₁₅—C(O)—NH—(CH₂)₃N⁺(CH₃)₂CH₂COO⁻
H₃C—(CH₂)₇—CH═CH—(CH₂)₇—C(O)—NH—(CH₂)₃N⁺(CH₃)₂CH₂COO—.

Particularly preferably, surfactants of formula (Bet-I) are used within narrower amount ranges. Preferred here are hair treatment agents as contemplated herein which contain, based on their weight, from about 0.25 to about 8 wt. %, more preferably from about 0.5 to about 7 wt. %, even more preferably from about 0.75 to about 6.5 wt. %, and in particular from about 1 to about 5.5 wt. %, of surfactant(s) of formula (Bet-I).

In addition to or instead of the ampho-surfactants of formula (Bet-I), the hair treatment agents as contemplated herein may particularly preferably contain, as amphoteric surfactants betaines of formula (Bet-II)

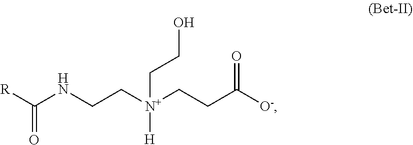

in which R represents a straight-chain or branched, saturated or mono or polyunsaturated alkyl or alkenyl functional group having from about 8 to about 24 carbon atoms.

In accordance with the INCI nomenclature, these surfactants are referred to as amphoacetates, the representatives derived from coconut fatty acids being preferred and referred to as cocoamphoacetates.

For technical reasons relating to the preparation thereof, surfactants of this type always also contain betaines of formula (Bet-IIa)

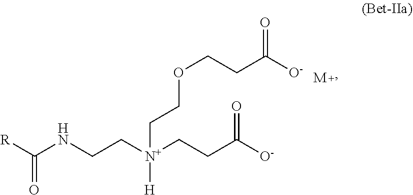

in which R represents a straight-chain or branched, saturated or mono or polyunsaturated alkyl or alkenyl functional group having from about 8 to about 24 carbon atoms, and M represents a cation.

In accordance with the INCI nomenclature, these surfactants are referred to as amphodiacetates, the representatives derived from coconut fatty acids being preferred and referred to as cocoamphodiacetates.

It is particularly preferable as contemplated herein to use surfactants of formula (Bet-II) that are a mixture of the following representatives:

H₃C—(CH₂)₇—C(O)—NH—(CH₂)₂NH⁺(CH₂CH₂OH)CH₂CH₂COO⁻
H₃C—(CH₂)₉—C(O)—NH—(CH₂)₂NH⁺(CH₂CH₂OH)CH₂CH₂COO⁻
H₃C—(CH₂)₁₁—C(O)—NH—(CH₂)₂NH⁺(CH₂CH₂OH)CH₂CH₂COO⁻
H₃C—(CH₂)₁₃—C(O)—NH—(CH₂)₂NH⁺(CH₂CH₂OH)CH₂CH₂COO⁻
H₃C—(CH₂)₁₅—C(O)—NH—(CH₂)₂NH⁺(CH₂CH₂OH)CH₂CH₂COO⁻
H₃C—(CH₂)₇—CH═CH—(CH₂)₇—C(O)—NH—(CH₂)₂NH⁺(CH₂CH₂OH)CH₂CH₂COO⁻

Particularly preferably, surfactants of formula (Bet-II) are used within narrower amount ranges. Preferred here are hair treatment agents as contemplated herein which contain, based on their weight, from about 0.25 to about 8 wt. %, more preferably from about 0.5 to about 7 wt. %, even more preferably from about 0.75 to about 6.5 wt. %, and in particular from about 1 to about 5.5 wt. %, of surfactant(s) of formula (Bet-II).

In summary, cosmetic agents as contemplated herein are preferred in which the functional group R in formulas (Bet-I) and (Bet-II) is selected from

H₃C—(CH₂)₇— H₃C—(CH₂)₉— H₃C—(CH₂)₁₁— H₃C—(CH₂)₁₃— H₃C—(CH₂)₁₅—H₃C—(CH₂)₇—CH═CH—(CH₂)₇— or mixtures thereof.

The hair treatment agents may contain nonionic surfactant(s).

Hair treatment agents which are preferred as contemplated herein contain, based on their weight, from about 0.3 to about 10 wt. %, preferably from about 0.5 to about 8 wt. %, more preferably from about 0.75 to about 6 wt. %, and in particular from about 1 to about 5 wt. %, of nonionic surfactant(s).

The hair treatment agents may contain cationic surfactant(s). As contemplated herein, it is possible to use cationic surfactants of the following types: quaternary ammonium compounds, esterquats, and amidoamines. Preferred quaternary ammonium compounds are ammonium halides, in particular chlorides and bromides, such as alkyltrimethylammonium chlorides, dialkyldimethyl ammonium chlorides and trialkylmethylammonium chlorides. The long alkyl chains of these surfactants preferably have from about 10 to about 18 carbon atoms, such as in cetyl trimethylammonium chloride, stearyl trimethylammonium chloride, distearyl dimethylammonium chloride, lauryl dimethylammonium chloride, lauryl dimethylbenzylammonium chloride, and tricetyl methylammonium chloride. The preferred cationic surfactants also include the imidazolium compounds known by the INCL names quaternium-27 and quaternium-83.

Particularly preferred hair treatment agents as contemplated herein contain as the cationic care substance, based on their weight, from about 0.05 to about 7.5 wt. %, preferably from about 0.1 to about 5 wt. %, particularly preferably from about 0.2 to about 3.5 wt. %, and in particular from about 0.25 to about 2.5 wt. %, of cationic surfactant(s) from the group of quaternary ammonium compounds and/or esterquats and/or amidoamines.

Particularly preferred hair treatment agents as contemplated herein contain, based on the weight of the agent, from about 0.05 to about 20 wt. %, preferably from about 0.1 to about 10 wt. %, more preferably from about 0.25 to about 8 wt. %, and in particular from about 0.5 to about 7 wt. %, of cationic surfactant(s), and preferably from about 0.05 wt. % to about 20 wt. %, more preferably from about 0.1 to about 10 wt. %, even more preferably from about 0.25 to about 8 wt. %, and in particular from about 0.5 to about 7 wt. %, of behenyl trimethyl ammonium chloride.

The agents as contemplated herein may contain at least one cationic polymer.

Cationic polymers increase the nourishing performance of the hair treatment agents as contemplated herein (in particular, the effectiveness of the agents as contemplated herein with respect to hair breakage).

Irrespective of which cationic polymer(s) is/are used, preferred hair treatment agents contain, based on the weight of the agent, from about 0.01 to about 3 wt. %, preferably from about 0.05 to about 2 wt. %, more preferably from about 0.1 to about 1.5 wt. %, and in particular from about 0.15 to about 0.8 wt. %, of cationic polymer(s).

In summary, hair treatment agents which are preferred as contemplated herein contain, based on the weight of the agent, from about 0.01 to about 3 wt. %, preferably from about 0.05 to about 2 wt. %, more preferably from about 0.1 to about 1.5 wt. %, and in particular from about 0.15 to about 0.8 wt. %, of cationic polymer(s), and preferably from about 0.01 wt. % to about 3 wt. %, more preferably from about 0.05 to about 2 wt. %, even more preferably from about 0.1 to about 1.5 wt. %, and in particular from about 0.15 to about 0.8 wt. %, of at least one polymer from the group of cationic cellulose polymers and/or cationic guar derivatives.

It has been found that alpha-substituted aldehydes can further enhance the effect of the agents as contemplated herein. In particular, significant increases in performance are observed with regard to the inner structural strengthening of keratin fibers and the prevention of washout.

Hair treatment agents which are preferred as contemplated herein additionally contain, based on the weight of the agent, from about 0.001 to about 20 wt. % of at least one alpha-substituted aldehyde.

Preferred hair treatment agents as contemplated herein contain, based on their weight, from about 0.01 to about 10 wt. %, preferably from about 0.05 to about 7.5 wt. %, more preferably from about 0.1 to about 6 wt. %, and in particular from about 0.15 to about 5 wt. %, of alpha-substituted aldehyde(s) of formula (I)

Y—CH(X)—CHO (I),

in which
X represents —OH, —Cl, —Br, —I, —O—(CH₂)ₙ—CH₃ where n=1, 2, 3 or 4, or —O—(CH₂)ₘ—OH where m=1, 2 or 3;
Y represents —H, —CH₃, H₃C—(CH₂)ₖ— where k=1, 2, 3, 4, 5, 6, 7 or 8, —OH, —(CH₂)ₚ—OH where p=1, 2, 3 or 4, or —CHO.

Aldehydes that are preferably to be used as contemplated herein are described on pages 31 to 34 of the priority document as numbers 1 to 176.

Very particularly preferred hair treatment agents as contemplated herein contain, based on their weight, from about 0.01 to about 10 wt. %, preferably from about 0.05 to about 7.5 wt. %, more preferably from about 0.1 to about 6 wt. %, and in particular from about 0.15 to about 5 wt. %, of at least one alpha-substituted aldehyde from the group

2-hydroxypropanal (X=—OH, Y=—CH₃):

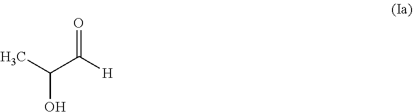

2-hydroxyhexanal (X=—OH, Y=H₃C—(CH₂)ₖ— where k=3):

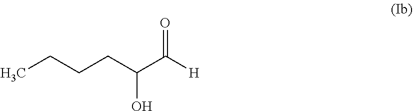

2-hydroxyhexanal (X=—OH, Y=H₃C—(CH₂)ₖ— where k=5):

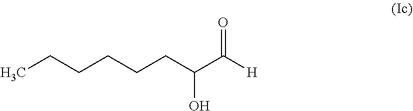

bromomalonaldehyde (X=Br, Y=—CH═):

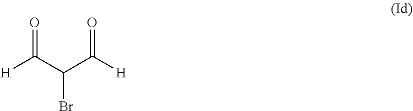

2-(2-hydroxyethoxy)acetaldehyde (X=—O—(CH₂)ₚ—OH where p=2, Y=—H):

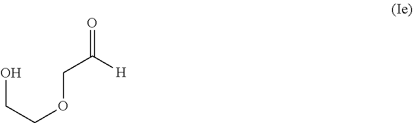

glyceraldehyde (X=—OH, Y=—(CH₂)ₚ—OH where p=1)

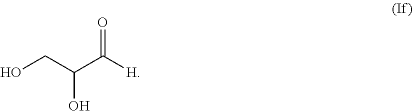

It has been found that succinimidyl esters can further enhance the effect of the agents as contemplated herein. In particular, significant increases in performance are observed with regard to the inner structural strengthening of keratin fibers and the prevention of washout.

Hair treatment agents which are preferred as contemplated herein additionally contain, based on the weight of the agent, from about 0.001 to about 10 wt. % of at least one succinimidyl ester.

Succinimidyl esters are esters of carboxylic acids having (optionally substituted) N-hydroxysuccinimide (NHS, IUPAC 1-hydroxy-2,5-pyrrolidinedione) and are also referred to as NHS esters. Hair treatment agents which are preferred as contemplated herein additionally contain, based on the weight of the agent, from about 0.01 to about 10 wt. %, preferably from about 0.05 to about 7.5 wt. %, more preferably from about 0.1 to about 6 wt. %, and in particular from about 0.15 to about 5 wt. %, of (a) succinimidyl ester(s) of formula (III)

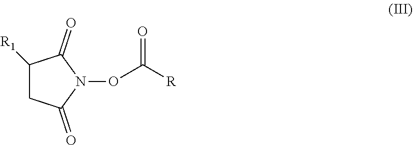

in which
R1 represents —H or an ionic group,
R represents an optionally substituted saturated or unsaturated, linear, branched or cyclic, aliphatic or aromatic hydrocarbon functional group having at least about 5 C atoms.

Suitable ionic groups R1 include both anionic groups such as phosphate, phosphonate, phosphinate, sulfate, sulfonate, sulfinate, sulfenate, oxysulfonate, carboxylate groups and cationic groups such as substituted or unsubstituted ammonium groups. Zwitterionic/betainic groups such as carboxybetaine or sulfobetaine groups are also possible.

In succinimidyl esters which are preferred as contemplated herein, R1 represents —H, —OSO₃⁻, —SO₃⁻, —SO₂⁻, —COO⁻, —NH₃⁺, —N(CH₃)H₂⁺, —N(CH₃)₂H⁺, —N(CH₃)₃⁺, —N⁺(CH₃)₂(CH₂)₂—COO⁻, or —N⁺(CH₃)₂(CH₂)₃—SO₃⁻.

Succinimidyl esters that are preferably to be used as contemplated herein are described on pages 37 to 39 of the priority document as numbers 1 to 72.

Very particularly preferred hair treatment agents as contemplated herein contain, based on their weight, from about 0.01 to about 10 wt. %, preferably from about 0.05 to about 7.5 wt. %, more preferably from about 0.1 to about 6 wt. %, and in particular from about 0.15 to about 5 wt. %, of at least one succinimidyl ester from the group

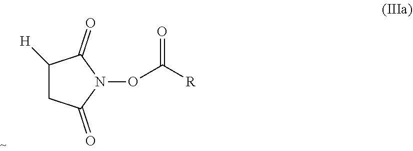

in which R represents -Ph or —(CH₂)ₙCH₃ where n=4, 5, 6, 7, 8, 9 or 10,

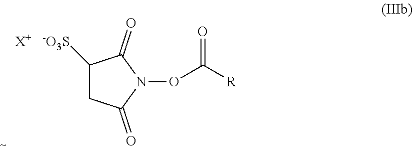

in which R represents -Ph or —(CH₂)ₙCH₃ where n=4, 5, 6, 7, 8, 9 or 10 and X represents H, a monovalent cation or the n-th part of an n-valent cation,

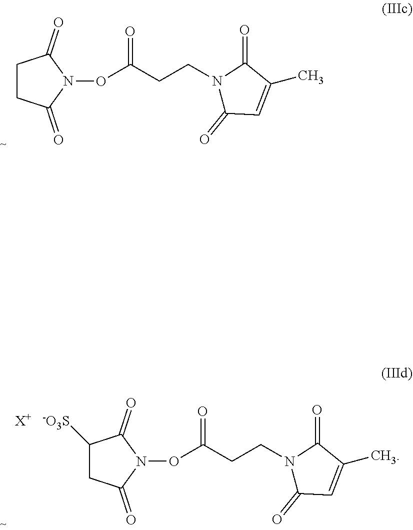

The hair treatment agents contain the above-described active ingredients preferably in a cosmetically acceptable carrier. Within the context of the present disclosure, this is understood to preferably mean an aqueous or aqueous-alcoholic carrier.

The cosmetic carrier preferably contains at least about 50 wt. %, more preferably at least about 60 wt. %, particularly preferably at least about 70 wt. %, and more particularly preferably at least about 75 wt. %, of water.

Furthermore, the cosmetic carrier may contain from about 0.01 to about 40 wt. %, preferably from about 0.05 to about 30 wt. %, and in particular from about 0.1 to about 20 wt. %, of at least one alcohol.

Suitable alcohols are, for example, ethanol, ethyl diglycol, 1-propanol, 2-propanol, isopropanol, 1,2-propylene glycol, glycerol, diglycerol, triglycerol, 1-butanol, 2-butanol, 1,2-butanediol, 1,3-butanediol, 1-pentanol, 2-pentanol, 1,2-pentanediol, 1,5-pentanediol, 1-hexanol, 2-hexanol, 1,2-hexanediol, 1,6-hexanediol, polyethylene glycols, sorbitol, sorbitan, benzyl alcohol, or mixtures of said alcohols.

Water-soluble alcohols are particularly preferred. Ethanol, 1,2-propylene glycol, glycerol, benzyl alcohol, and mixtures of said alcohols are particularly preferred.

It is advantageous for very good (scalp) skin compatibility of the hair treatment agents as contemplated herein if said agents have a slightly acidic pH.

It has been found that the agents as contemplated herein have particularly good skin compatibility and mildness in a pH range of from about 4.2 to about 5.8.

In a first preferred embodiment, the hair treatment agents as contemplated herein therefore preferably have a pH in the range of from about 4.2 to about 5.8, more preferably from about 4.3 to about 5.6, particularly preferably from about 4.4 to about 5.5, extremely preferably from about 4.5 to about 5.4, and particularly preferably from about 4.7 to about 5.3.

The hair treatment agents as contemplated herein may contain vegetable oils, vegetable butters, and/or vegetable waxes. These vegetable oil components give the hair improved combability and stylability and increase hair shine.

Suitable vegetable oil components include natural (vegetable) oils and/or butters which typically contain triglycerides and mixtures of triglycerides.

Preferred natural oils are coconut oil, (sweet) almond oil, walnut oil, peach kernel oil, apricot kernel oil, argan oil, avocado oil, tea tree oil, soybean oil, sesame oil, sunflower oil, tsubaki oil, evening primrose oil, rice bran oil, palm kernel oil, mango kernel oil, marula oil, cuckoo flower oil, thistle oil, macadamia nut oil, grape seed oil, amaranth seed oil, bamboo oil, olive oil, wheat germ oil, pumpkin seed oil, mallow oil, hazelnut oil, safflower oil, canola oil, sasanqua oil, jojoba oil, rambutan oil, cocoa butter, and/or shea butter.

Carnauba wax, beeswax, and/or candelilla wax can preferably be used as suitable natural or vegetable waxes.

Particularly preferred vegetable oil components are (sweet) almond oil, peach kernel oil, apricot kernel oil, amaranth seed oil, argan oil, olive oil, jojoba oil, cocoa butter, and/or shea butter. Apricot kernel oil, argan oil, olive oil, and/or jojoba oil are particularly preferred.

In a preferred embodiment, the hair treatment agents of the present disclosure preferably contain coconut oil, (sweet) almond oil, walnut oil, peach kernel oil, apricot kernel oil, argan oil, avocado oil, tea tree oil, soybean oil, sesame oil, sunflower oil, tsubaki oil, evening primrose oil, rice bran oil, palm kernel oil, mango kernel oil, marula oil, cuckoo flower oil, thistle oil, macadamia nut oil, grape seed oil, amaranth seed oil, bamboo oil, olive oil, wheat germ oil, pumpkin seed oil, mallow oil, hazelnut oil, safflower oil, canola oil, sasanqua oil, jojoba oil, rambutan oil, cocoa butter, and/or shea butter.

Within this embodiment, it is particularly preferable if the hair treatment agents as contemplated herein contain (sweet) almond oil, peach kernel oil, apricot kernel oil, amaranth seed oil, argan oil, olive oil, jojoba oil, cocoa butter, and/or shea butter.

The weight proportion of the at least one vegetable oil, vegetable butter, and/or vegetable wax in terms of the total weight of the hair treatment agents as contemplated herein is preferably from about 0.02 to about 2.50 wt. %, more preferably from about 0.03 to about 2.00 wt. %, particularly preferably from about 0.04 to about 1.50 wt. %, and in particular from about 0.05 to about 1.00 wt. %.

In addition to the aforementioned essential and optional components, the hair treatment agents as contemplated herein may contain, in a further preferred embodiment for further increasing the nourishing properties of the agents, at least one further active hair-conditioning ingredient, which can be selected from the group of
protein hydrolyzates,
vitamins,
plant extracts, and/or
glycerol.

Suitable protein hydrolyzates are understood to mean product mixtures that can be obtained by acid, base, or enzyme-catalyzed degradation of proteins.

Protein hydrolyzates of plant, animal, and/or marine origin can be used.

Animal protein hydrolyzates are, for example, elastin, collagen, keratin, silk, and milk protein hydrolyzates, which can also be present in the form of salts. Such products are marketed, for example, under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex), and Kerasol® (Croda).

Protein hydrolyzates of plant origin, e.g. soybean, almond, rice, pea, potato and wheat protein hydrolyzates, are preferred. Such products are available, for example, under the tradenames Gluadin® (Cognis), DiaMin® (Diamalt), Lexein® (Inolex), and Crotein® (Croda). Cationized protein hydrolyzates can also be used, it being possible for the underlying protein hydrolyzate to originate from animals, for example from collagen, milk or keratin, from plants, for example from wheat, corn, rice, potatoes, soybeans or almonds, from marine life forms, for example from fish collagen or algae, or from biotechnologically obtained protein hydrolyzates. The protein hydrolyzates forming the basis of the cationic derivatives may be obtained from the corresponding proteins by chemical, in particular alkaline or acidic, hydrolysis, by enzymatic hydrolysis, and/or by a combination of both types of hydrolysis. The hydrolysis of proteins usually results in a protein hydrolyzate having a molecular weight distribution of approximately 100 daltons up to several thousand daltons. Cationic protein hydrolyzates are preferred whose underlying protein portion has a molecular weight of from about 100 to about 25,000 daltons, preferably from about 250 to about 5000 daltons. Furthermore, cationic protein hydrolyzates are understood to mean quaternized amino acids and mixtures thereof. Quaternization of the protein hydrolyzates or of the amino acids is often carried out by employing quaternary ammonium salts such as N,N-dimethyl-N-(n-alkyl)-N-(2-hydroxy-3-chloro-n-propyl)-ammonium halides. Furthermore, the cationic protein hydrolyzates can also be derivatized even further. Typical examples of cationic protein hydrolyzates and derivatives are the commercially available products known under the following INCI names: cocodimonium hydroxypropyl hydrolyzed collagen, cocodimonium hydroxypropyl hydrolyzed casein, cocodimonium hydroxypropyl hydrolyzed collagen, cocodimonium hydroxypropyl hydrolyzed hair keratin, cocodimonium hydroxypropyl hydrolyzed keratin, cocodimonium hydroxypropyl hydrolyzed rice protein, cocodimonium hydroxypropyl hydrolyzed silk, cocodimonium hydroxypropyl hydrolyzed soy protein, cocodimonium hydroxypropyl hydrolyzed wheat protein, cocodimonium hydroxypropyl silk amino acids, hydroxypropyl arginine lauryl/myristyl ether HCl, hydroxypropyltrimonium gelatin, hydroxypropyltrimonium hydrolyzed casein, hydroxypropyltrimonium hydrolyzed collagen, hydroxypropyltrimonium hydrolyzed conchiolin protein, hydroxypropyltrimonium hydrolyzed keratin, hydroxypropyltrimonium hydrolyzed rice bran protein, hydroxypropyltrimonium hydrolyzed silk, hydroxypropyltrimonium hydrolyzed soy protein, hydroxypropyl hydrolyzed vegetable protein, hydroxypropyltrimonium hydrolyzed wheat protein, hydroxypropyltrimonium hydrolyzed wheat protein/siloxysilicate, laurdimonium hydroxypropyl hydrolyzed soy protein, laurdimonium hydroxypropyl hydrolyzed wheat protein, laurdimonium hydroxypropyl hydrolyzed wheat protein/siloxysilicate, lauryldimonium hydroxypropyl hydrolyzed casein, lauryldimonium hydroxypropyl hydrolyzed collagen, lauryldimonium hydroxypropyl hydrolyzed keratin, lauryldimonium hydroxypropyl hydrolyzed silk, lauryldimonium hydroxypropyl hydrolyzed soy protein, steardimonium hydroxypropyl hydrolyzed casein, steardimonium hydroxypropyl hydrolyzed collagen, steardimonium hydroxypropyl hydrolyzed keratin, steardimonium hydroxypropyl hydrolyzed rice protein, steardimonium hydroxypropyl hydrolyzed silk, steardimonium hydroxypropyl hydrolyzed soy protein, steardimonium hydroxypropyl hydrolyzed vegetable protein, steardimonium hydroxypropyl hydrolyzed wheat protein, steartrimonium hydroxyethyl hydrolyzed collagen, quaternium-76 hydrolyzed collagen, quaternium-79 hydrolyzed collagen, quaternium-79 hydrolyzed keratin, quaternium-79 hydrolyzed milk protein, quaternium-79 hydrolyzed silk, quaternium-79 hydrolyzed soy protein, quaternium-79 hydrolyzed wheat protein.

The weight proportion of the protein hydrolyzate(s) in terms of the total weight of the hair treatment agents is preferably from about 0.01 to about 5 wt. %, more preferably from about 0.025 to about 3 wt. %, and in particular from about 0.05 to about 2 wt. %.

Irrespective of the source (plant, animal, marine, etc.), protein hydrolyzates contain, depending on the degree of hydrolysis, individual amino acids, oligopeptides, and optionally polypeptides.

Preferred hair treatment agents as contemplated herein are at least about 0.1 wt. %, preferably at least about 0.5 wt. %, particularly preferably at least about 1 wt. %, more preferably at least about 2.5 wt. %, even more preferably at least about 5 wt. %, and in particular at least about 10 wt. %, of all the keratin peptides contained in the agent have the amino acid sequence Glu-Glu-Glu.

Hair treatment agents as contemplated herein that are more preferred are at least about 0.1 wt. %, preferably at least about 0.5 wt. %, particularly preferably at least about 1 wt. %, more preferably at least about 2.5 wt. %, even more preferably at least about 5 wt. %, and in particular at least about 10 wt. %, of all the keratin peptides contained in the agent have the amino acid sequence Glu-Glu-Glu-Ile.

Hair treatment agents as contemplated herein that are even more preferred are at least about 0.1 wt. %, preferably at least about 0.5 wt. %, particularly preferably at least about 1 wt. %, more preferably at least about 2.5 wt. %, even more preferably at least about 5 wt. %, and in particular at least about 10 wt. %, of all the keratin peptides contained in the agent have the amino acid sequence Tyr-Glu-Glu-Glu.

Particularly preferred hair treatment agents as contemplated herein are at least about 0.1 wt. %, preferably at least about 0.5 wt. %, particularly preferably at least about 1 wt. %, more preferably at least about 2.5 wt. %, even more preferably at least about 5 wt. %, and in particular at least about 10 wt. %, of all the keratin peptides contained in the agent have the amino acid sequence Tyr-Glu-Glu-Glu-Ile.

Hair treatment agents as contemplated herein that are very particularly preferred are at least about 0.1 wt. %, preferably at least about 0.5 wt. %, particularly preferably at least about 1 wt. %, more preferably at least about 2.5 wt. %, even more preferably at least about 5 wt. %, and in particular at least about 10 wt. %, of all the keratin peptides contained in the agent have the amino acid sequence Tyr-Glu-Glu-Glu-Ile-Arg.

Hair treatment agents as contemplated herein that are even more preferred are at least about 0.1 wt. %, preferably at least about 0.5 wt. %, particularly preferably at least about 1 wt. %, more preferably at least about 2.5 wt. %, even more preferably at least about 5 wt. %, and in particular at least about 10 wt. %, of all the keratin peptides contained in the agent have the amino acid sequence Tyr-Glu-Glu-Glu-Ile-Arg-Val.

Hair treatment agents as contemplated herein that are particularly preferred are at least about 0.1 wt. %, preferably at least about 0.5 wt. %, particularly preferably at least about 1 wt. %, more preferably at least about 2.5 wt. %, even more preferably at least about 5 wt. %, and in particular at least about 10 wt. %, of all the keratin peptides contained in the agent have the amino acid sequence Tyr-Glu-Glu-Glu-Ile-Arg-Val-Leu.

The aforementioned conditions relate to the total content of peptides originating from keratin materials in the agents as contemplated herein. In addition to the oligopeptides of keratin origin, further peptides and/or protein hydrolyzates can of course be used, for example from other native sources. For example, the additional use of wheat protein hydrolyzates is preferred.

Suitable vitamins are understood preferably to be the following vitamins, provitamins, and vitamin precursors, and derivatives thereof:
Vitamin A: the group of substances designated as vitamin A includes retinol (vitamin A₁) and also 3,4-didehydroretinol (vitamin A₂). β-carotene is the provitamin of retinol. Suitable vitamin A components are for example vitamin A acid and esters thereof, vitamin A aldehyde and vitamin A alcohol and esters thereof such as the palmitate and the acetate.
Vitamin B: the vitamin B group or the vitamin B complex includes, inter alia,
   vitamin B₁ (thiamine)
   vitamin B₂ (riboflavin)
   vitamin B₃. The compounds nicotinic acid and nicotinamide (niacinamide) are often included under this designation.
   vitamin B₅ (panthothenic acid and panthenol). Panthenol is preferably used within the context of this group. Derivatives of panthenol which can be used are in particular the esters and ethers of panthenol, pantolactone as well as cationically derivatized panthenols. Individual representatives are, for example, panthenol triacetate, panthenol monoethyl ether, and the monoacetate thereof, as well as cationic panthenol derivatives.
   Vitamin B₆ (pyridoxine as well as pyridoxamine and pyridoxal).
Vitamin C (ascorbic acid): the use in the form of the palmitic acid ester, glucosides, or phosphates can be preferred. The use in combination with tocopherols can likewise be preferred.
Vitamin E (tocopherols, in particular α-tocopherol).
Vitamin F: the term “vitamin F” is conventionally understood to mean essential fatty acids, in particular linoleic acid, linolenic acid, and arachidonic acid.
Vitamin H: The compound (3aS,4S, 6aR)-2-oxohexahydrothienol[3,4-d]-imidazole-4-valeric acid is designated as vitamin H, for which the trivial name biotin has become accepted.

Vitamins, provitamins, and vitamin precursors from the groups A, B, E, and H are particularly preferred. Particularly preferred are nicotinamide, biotin, pantolactone, and/or panthenol.

The weight proportion of the vitamin(s), vitamin derivative(s), and/or vitamin precursor(s) in terms of the total weight of the hair treatment agents is preferably from about 0.001 to about 2 wt. %, particularly preferably from about 0.005 to about 1 wt. %, and in particular from about 0.01 to about 0.5 wt. %.

Suitable plant extracts are to be understood to mean extracts that can be prepared from all the parts of a plant. These extracts are typically prepared by extraction of the entire plant. It may also be preferable in some cases, however, to prepare the extracts solely from the flowers and/or leaves of the plant. Particularly suitable are the extracts from green tea, oak bark, stinging nettle, witch hazel, hops, chamomile, burdock root, horsetail, whitethorn, lime blossom, lychee, almond, aloe vera, pine needles, horse chestnut, sandalwood, juniper, coconut, mango, apricot, lemon, wheat, kiwi, melon, orange, grapefruit, sage, rosemary, birch, mallow, cuckoo flower, wild thyme, yarrow, thyme, melissa, restharrow, coltsfoot, marshmallow, ginseng, ginger root, Echinacea purpurea, Olea europaea, Boerhavia diffusa roots, Foeniculum vulgaris, and Apium graveolens.

The extracts from green tea, stinging nettle, witch hazel, chamomile, aloe vera, ginseng, Echinacea purpurea, Olea europaea, and/or Boerhavia diffusa roots are particularly preferred for use in the compositions as contemplated herein.

The plant extracts can be used in the hair treatment agents as contemplated herein (based on the total weight of the agents) preferably in an amount of from about 0.01 to about 10 wt. %, more preferably from about 0.05 to about 7.5 wt. %, and in particular from about 0.1 to about 5 wt. %.

Glycerol can be added to the hair cleaning and care agents separately in an amount of up to about 10 wt. % (based on the total weight of the agent). However, it may also be a component of the above-mentioned aqueous-alcoholic carrier.

It has been found that the hair treatment agents as contemplated herein are also suitable for use as an anti-dandruff preparation.

The total weight of anti-dandruff agents in terms of the total weight of the hair treatment agents can preferably be from about 0.01 to about 10 wt. %, more preferably from about 0.025 to about 7.5 wt. %, particularly preferably from about 0.05 to about 5 wt. %, and in particular from about 0.075 to about 3 wt. %.

Suitable anti-dandruff active ingredients can be selected from piroctone olamine, climbazole, zinc pyrithione, ketoconazole, salicylic acid, sulfur, selenium sulfide, tar preparations, undecenoic acid derivatives, burdock root extracts, poplar extracts, stinging nettle extracts, walnut shell extracts, birch extracts, willow bark extracts, rosemary extracts, and/or arnica extracts.

Climbazole, zinc pyrithione, and piroctone olamine are preferred.

Other active substances, auxiliary substances and additives that can preferably be contained in the hair treatment agents as contemplated herein are, for example:
humectants,
perfumes,
UV filters,
thickening agents such as gelatins or plant gums, for example agar-agar, guar gum, alginates, xanthan gum, gum arabic, karaya gum, locust bean flour, linseed gums, dextrans, cellulose derivatives, for example methyl cellulose, hydroxyalkyl cellulose and carboxymethyl cellulose, starch fractions and derivatives, such as amylose, amylopectin and dextrins, clays and sheet silicates, such as bentonite or fully synthetic hydrocolloids such as polyvinyl alcohol, the Ca, Mg or Zn soaps,
structurants such as maleic acid and lactic acid,
dimethyl isosorbide,
cy clodextrins,
fiber-structure-improving active ingredients, in particular mono, di, and oligosaccharides such as glucose, galactose, fructose, fruit sugar, and lactose,
dyes for coloring the agent,
active ingredients such as bisabolol and/or allantoin,
complexing agents such as EDTA, NTA, β-alanine diacetic acid and phosphonic acids,
ceramides. Ceramides are understood to mean N-acylsphingosine (fatty acid amides of sphingosine) or synthetic analogs of such lipids (so-called pseudo-ceramides),
propellants such as propane-butane mixtures, N₂O, dimethyl ether, CO₂ and air,
antioxidants,
additional viscosity regulators, such as salts (NaCl).

The agents as contemplated herein can be formulated as what are referred to as rinse-off products, i.e. are rinsed out of the hair again after a specific contact time. This contact time is preferably less than an hour, i.e. the consumer preferably does not leave the products in the hair until the next time the hair is washed.

A further subject of the present disclosure is therefore a method for treating hair in which an agent as contemplated herein is applied to dry or wet hair, is left there for a period of from about 10 to about 300 seconds, and is rinsed out thereafter.

The agents as contemplated herein can also be formulated as what are referred to as leave-on products, i.e. are not rinsed out of the hair, but are instead left there until the next time the hair is washed. A further subject of the present disclosure is therefore a method for treating hair in which an agent as contemplated herein is applied to dry or wet hair and is left there until the next time the hair is washed.

The agents as contemplated herein lead to a significantly increased strengthening of the internal and external hair structure. A further subject of the present disclosure is therefore the use of agents as contemplated herein for strengthening the hair structure, in particular the internal hair structure.

Within the meaning of the present disclosure, “structure strengthening” is understood to mean a reduction in the damage to keratin fibers caused by a wide range of influences. Re-establishing the natural strength, for example, plays a significant role in this. Restructured fibers are distinguished, for example, by improved shine, by an improved feel, and by easier combability. In addition, they have optimized strength and elasticity. Successful structure strengthening or restructuring can be verified physically as an increase in the melting point in comparison with damaged fibers.

The agents as contemplated herein also lead to considerably increased stability of artificial colors against the washout of color. Chemically colored hair can thus be washed much more frequently using the agents as contemplated herein, without this leading to undesired bleeding or fading of the color.

A further subject of the present disclosure is therefore the use of agents as contemplated herein for reducing the washout of color from chemically colored hair.

The statements made about the agents as contemplated herein apply, mutatis mutandis, to particularly preferred embodiments of the methods as contemplated herein and to the uses as contemplated herein.

### Examples

### Hair Shampoo:

| | 1 | 2 | 3 | 4 | 5 | 6 |
|---|---|---|---|---|---|---|
| Sodium laureth sulfate | 10 | 10 | 10 | 10 | 10 | 10 |
| Cocoamidopropyl betaine | 4.0 | 4.0 | 4.0 | 4.0 | 4.0 | 4.0 |
| Ammonium lauryl sulfate | 4.0 | 4.0 | 4.0 | 4.0 | 4.0 | 4.0 |
| Polyquaternium-10 | 0.3 | 0.3 | 0.3 | 0.3 | 0.3 | 0.3 |
| Cocamide MEA | 0.8 | 0.8 | 0.8 | 0.8 | 0.8 | 0.8 |
| PEG-7 glyceryl cocoate | 1.0 | 1.0 | 1.0 | 1.0 | 1.0 | 1.0 |
| Nicotinamide | 0.3 | 0.3 | 0.3 | 0.3 | 0.3 | 0.3 |
| Citric acid | 0.8 | 0.8 | 0.8 | 0.8 | 0.8 | 0.8 |
| Panthenol | 0.5 | 0.5 | 0.5 | 0.5 | 0.5 | 0.5 |
| Tetrasodium glutamate | 1.0 | 0.1 | — | — | — | — |
| diacetate | | | | | | |
| Pentasodium | — | — | 1.0 | 0.1 | — | — |
| diethylenetriaminepentaacetate | | | | | | |
| Tetrasodium iminodisuccinate | — | — | — | — | 1.0 | 0.1 |
| Calcium lactate | 1.5 | 1.5 | 1.5 | 1.5 | 1.5 | 1.5 |
| Laureth-2 | 1.2 | 1.2 | 1.2 | 1.2 | 1.2 | 1.2 |
| Sodium chloride | 1.3 | 1.3 | 1.3 | 1.3 | 1.3 | 1.3 |
| Water, preservative and | up to | up to | up to | up to | up to | up to |
| optionally perfume oils | 100 | 100 | 100 | 100 | 100 | 100 |

### Hair Conditioner:

| | 1 | 2 | 3 | 4 | 5 | 6 |
|---|---|---|---|---|---|---|
| Quaternium-87 | 0.75 | 0.75 | 0.75 | 0.75 | 0.75 | 0.75 |
| Glycol distearate | 1.0 | 1.0 | 1.0 | 1.0 | 1.0 | 1.0 |
| Cetearyl alcohol | 5.0 | 5.0 | 5.0 | 5.0 | 5.0 | 5.0 |
| Guar hydroxypropyltrimonium | 0.2 | 0.2 | 0.2 | 0.2 | 0.2 | 0.2 |
| chloride | | | | | | |
| Polyquaternium-37 | 0.4 | 0.4 | 0.4 | 0.4 | 0.4 | 0.4 |
| Magnesium sulfate | 0.5 | 0.5 | 0.5 | 0.5 | 0.5 | 0.5 |
| Shea butter (INCI: | 1.5 | 1.5 | 1.5 | 1.5 | 1.5 | 1.5 |
| butyrospermum parkii (shea) | | | | | | |
| butter) | | | | | | |
| Behenoyl | 1.5 | 1.5 | 1.5 | 1.5 | 1.5 | 1.5 |
| PG-trimoniumchloride | | | | | | |
| Distearoylethyl | 0.3 | 0.3 | 0.3 | 0.3 | 0.3 | 0.3 |
| hydroxyethylmonium | | | | | | |
| methosulfate | | | | | | |
| Behentrimonium chloride | 0.4 | 0.4 | 0.4 | 0.4 | 0.4 | 0.4 |
| Lactic acid | 0.3 | 0.3 | 0.3 | 0.3 | 0.3 | 0.3 |
| Tetrasodium glutamate | 0.8 | 0.1 | — | — | — | — |
| diacetate | | | | | | |
| Pentasodium | — | — | 0.8 | 0.1 | — | — |
| diethylenetriaminepentaacetate | | | | | | |
| Tetrasodium iminodisuccinate | — | — | — | — | 0.8 | 0.1 |
| Dimethicone | 0.1 | 0.1 | 0.1 | 0.1 | 0.1 | 0.1 |
| Water, preservative, | up to | up to | up to | up to | up to | up to |
| accompanying substances and | 100 | 100 | 100 | 100 | 100 | 100 |
| optionally perfume oils | | | | | | |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A hair treatment agent containing, based on its weight,
a) from about 0.001 to about 20 wt. % of complexing agents selected from the group consisting of
a. tetrasodium-N,N-bis(carboxylatomethyl)-L-glutamate (tetrasodium glutamate diacetate, GLDA) 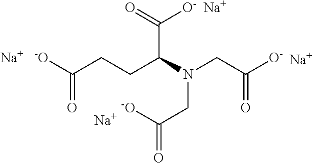
b. pentasodium diethylenetriaminepentaacetate (DTPA) 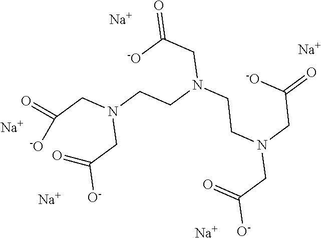
c. tetrasodium iminodisuccinate (IDS) 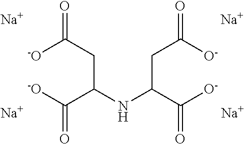
and either
b) from about 0.05 to about 0.5 mol/kg of at least one divalent salt of a monovalent organic acid or
c) from about 0.01 to about 0.5 mol/kg of at least one monovalent organic acid (c1), and from about 0.01 to about 0.1 mol/kg of at least one divalent inorganic salt (c2).

2. The hair treatment agent according to claim 1, comprising, based on its weight, from about 0.005 to about 15 wt. %, of tetrasodium-N,N-bis(carboxylatomethyl)-L-glutamate (tetrasodium glutamate diacetate, GLDA).

3. The hair treatment agent according to claim 1, comprising, based on its weight, from about 0.005 to about 15 wt. %, of pentasodium diethylenetriaminepentaacetate (DTPA).

4. The hair treatment agent according to claim 1, it comprising, based on its weight, from about 0.005 to about 15 wt. %, of tetrasodium iminodisuccinate (IDS).

5. The hair treatment agent according to claim 1, it, comprising
as component b), salts selected from alkaline-earth metal salts and zinc salts of formic acid, acetic acid, glycolic acid, gluconic acid, lactic acid, mandelic acid or glyoxylic acid, or,
as component (c1), formic acid, acetic acid, glycolic acid, gluconic acid, lactic acid, mandelic acid or glyoxylic acid, and, as component (c2), an alkaline-earth metal halide, alkaline-earth metal hydroxide, alkaline-earth metal sulfate, alkaline-earth metal oxide, zinc halide, zinc hydroxide, zinc sulfate, or zinc oxide.

6. The hair treatment agent according to claim 1, it, comprising
as component b), calcium lactate, or,
as component (c1), lactic acid, and, as component (c2), calcium chloride or calcium hydroxide.

7. The hair treatment agent according to claim 1, comprising
from about 0.04 to about 0.40 mol/kg, of salt b) or
from about 0.02 to about 0.40 mol/kg, of the monovalent organic acid (c1), and from about 0.04 to about 0.10 mol/kg, of the divalent organic acid (c2).

8. The hair treatment agent according to claim 1, having a pH in the range of from about 3.0 to about 5.0.

9. A method for treating hair, comprising applying an agent according to claim 1 to dry or wet hair, leaving the applied agent on the hair for a period of ten seconds or longer, and rinsing the agent out thereafter.

10. The method according to claim 9, comprising applying the agent to dry or wet hair and leaving it there until the next time the hair is washed.

11. A method of reducing washout of color from chemically colored hair comprising applying a composition according to claim 1 to the hair.

12. The method according to claim 9, comprising applying the agent to dry or wet hair, leaving the applied agent on the hair for a period of from about 10 to about 300 seconds, and rinsing the agent out thereafter.

13. The hair treatment agent according to claim 1, comprising calcium lactate.

14. The hair treatment agent according to claim 1, comprising calcium gluconate.

15. The hair treatment agent according to claim 1, further comprising an anti-dandruff active ingredient.

16. The hair treatment agent according to claim 1, further comprising an amino-functional silicone.
